(19) United States
(12) Patent Application Publication
(10) Pub. No.: US 2026/0334819 A1
(43) Pub. Date: Oct. 29, 2026

(54) AI-ENHANCED MUSIC-BASED MAGNETIC RESPIRATORY THERAPY SYSTEM

(71) Applicant: UNIVERSITY OF SOUTH FLORIDA, Tampa, FL (US)

(72) Inventors: Manh-Huong PHAN, Tampa, FL (US); Dang NGUYEN, Tampa, FL (US); Kee Young HWANG, Tampa, FL (US)

(21) Appl. No.: 19/660,149

(22) Filed: Apr. 27, 2026

Related U.S. Application Data
(60) Provisional application No. 63/794,843, filed on Apr. 25, 2025.

(51) Int. Cl. A61B 5/113; A61B 5/00; A61M 21/00

(52) U.S. Cl. CPC A61B 5/1135; A61B 5/486; A61M 21/00; A61M 2021/0027

(57) ABSTRACT

Systems and methods for music-modulated respiratory therapy are disclosed. A non-contact sensor detects respiratory motion of a user based on changes in magnetic flux produced by a permanent magnet positioned on the user's chest or clothing relative to a fixed magnetic sensor. A controller receives respiratory motion data from the non-contact sensor, extracts respiratory features including breathing rate, breathing uniformity, breathing amplitude, and spectral features, and determines the user's respiratory pattern. The controller compares the respiratory pattern against target or reference values derived from user information to identify deviations from an optimal respiratory condition. Based on identified deviations, the controller generates feedback signals to adjust parameters of a music source output, including tempo, volume, rhythm, pitch, or music selection, to modulate the user's breathing toward the optimal condition. Machine learning models may classify the user's respiratory state and predict music parameter adjustments. The system may detect anomalous breathing patterns and generate alerts when music-based intervention is insufficient.

### CROSS REFERENCE TO RELATED APPLICATIONS

This application claims priority to U.S. provisional patent application number 63/794,843, filed on Apr. 25, 2025, the entire contents of which are incorporated herein by reference.

### BACKGROUND

Various respiratory disturbances may occur during sleep, including sleep apnea, irregular breathing amplitude, and abnormal breathing rates. Such disturbances may be associated with conditions affecting the cardiorespiratory system, including lung disease, heart disease, and post-COVID-19 respiratory complications. Existing monitoring approaches, such as wearable consumer devices that measure blood oxygen saturation or heart rate, may not adequately capture the respiratory motion dynamics that characterize these disturbances.

During sleep, the medulla oblongata assumes primary responsibility for regulating respiratory rhythm, operating autonomously and independently of conscious control. Conventional approaches to respiratory therapy and rehabilitation may require active patient participation, clinical supervision, or cumbersome equipment, limiting their suitability for continuous or overnight use. Contact-based respiratory sensing techniques may present limitations in comfort and real-time performance in sleep environments, where sensor attachment may disturb the user or become displaced during movement.

### BRIEF DESCRIPTION OF THE DRAWINGS

The foregoing features of embodiments will be more readily understood by reference to the following detailed description, taken with reference to the accompanying drawings, in which:

FIG. 1 is a diagram illustrating an example system architecture 100 for a music-modulated respiratory therapy (MMRT) system according to some examples, showing user devices 105 (including user device 105a and user device 105c) connected via communication links 120 to cloud infrastructure 110, with a non-contact sensor 130 monitoring a user 134 via a wireless signal 132 and communicating with an MMRT system 140 comprising an I/O component 142 and a data analyzation component 144.

FIG. 2 is a diagram illustrating an example system 200 showing an MMRT system 240 deployed in a bedroom or sleep environment, with a non-contact sensor 230 monitoring a user 234 via wireless signal 232, a communication link 210, and providing music output 212 to the user according to some examples.

FIG. 3 is a flow diagram illustrating an example system flow 300 for the MMRT system, showing a respiratory monitoring component 310 comprising a sensing component 312, a respiratory motion mapping component 314, and an extraction component 316, which feeds a data analyzation component 318 that generates feedback 320 to a music source 322 to adjust a tune 324 according to some examples.

FIG. 4 is a block diagram illustrating an example system schematic 400 for an AI-assisted MMRT system, showing testing subjects 410 organized into demographic groups, Group I 422 (subjects under 19 years of age), Group II 424 (subjects between 19 and 59 years of age), and Group III 426 (subjects over 60 years of age), interacting with a respiratory sensor (RS) 418, an AI-assisted controller (AI-C) 420, a machine learning (ML) component 416, a reference data library 414, and a music source (MS) 412 according to some examples.

FIG. 5 is a diagram illustrating an example computing environment 500 for an MMRT system 505, showing a network 510, a database 512, network connections 520, AI agents 530, memory 535 storing code 545, a communications component 555, an input/output controller 565, a data analyzation component 570, and processors 575 according to some examples.

FIG. 6 is a flow diagram illustrating an example method flow for operating the MMRT system according to some examples.

### DETAILED DESCRIPTION

Described herein are systems and methods directed to music-modulated respiratory therapy (MMRT), and more particularly to contactless magnetic respiratory monitoring systems integrated with adaptive, AI-guided music therapy for real-time modulation of breathing patterns, detection of respiratory anomalies, and longitudinal personal respiratory health monitoring.

Examples of the described technology provide systems and methods for monitoring respiratory motion of a user in real time and providing adaptive music-based feedback designed to modulate the user's breathing toward an optimal or target condition. A system according to the disclosure may include a sensor component, a processing and control component, a music output component, and a reference data component. The sensor component may detect respiratory motion contactlessly, generating respiratory motion data that the processing and control component analyzes to extract respiratory features. Based on those features, the processing and control component may generate feedback signals that adjust music parameters in real time. The reference data component may provide population-level and individual-level data used by the processing and control component to determine appropriate target conditions and music adjustments for a given user.

Processes and algorithms described below are not limiting of the scope of this disclosure, can be combined in various configurations (in whole or in part), and may be adapted to replace, complement, and/or fit with attributes and needs of different aspects disclosed herein. Systems and methods of the present disclosure may be deployed in a variety of contexts, including, without limitation, sleep monitoring and therapy, respiratory rehabilitation, clinical monitoring, and general wellness applications. Other implementations within the scope of the claims will be apparent from the description herein.

In various examples, the processing and control component may incorporate one or more machine learning models trained on respiratory data from a plurality of subjects, enabling the processing and control component to learn associations between respiratory features and subject characteristics, including, for example, age group and health status, to classify a user's respiratory state, and to predict appropriate music parameter adjustments. In certain examples, data collected during each user session may be stored and used to refine the machine learning models or to build a longitudinal respiratory health profile for the individual user.

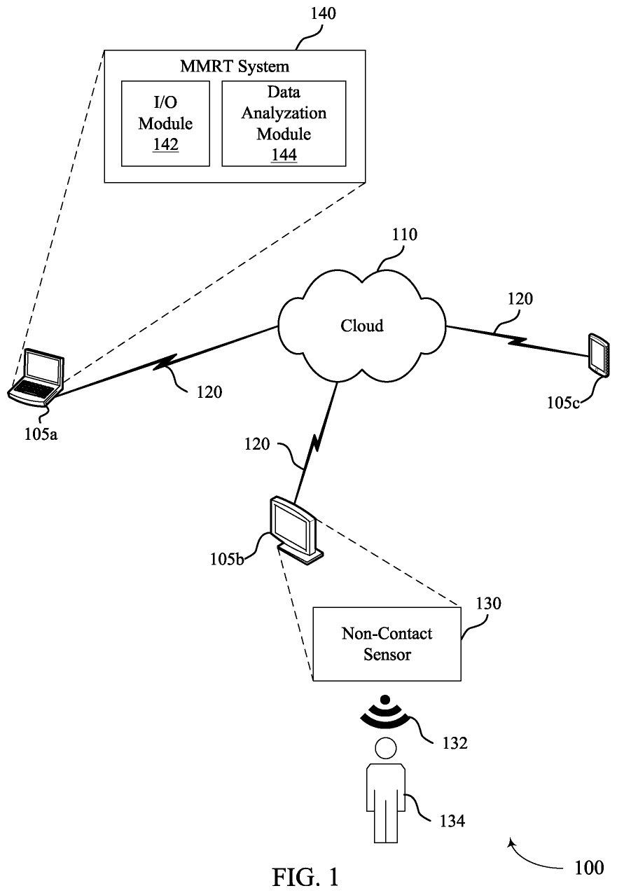

FIG. 1 is a schematic diagram of an example computing environment that supports a music-modulated respiratory therapy (MMRT) device in accordance with one or more aspects of the present disclosure. The network environment 100 may include one or more computing devices 105-a through 105-c (collectively referred to as computing devices 105) and at least one network 110. The at least one network 110 may be in communication with the one or more computing devices 105 via one or more communication links 120. The one or more computing devices 105 may be in communication with each other via the at least one network 110 or via direct peer-to-peer communications, for example.

The one or more computing devices 105 may be any type of computing device, including, for example, a desktop computer, a laptop computer, a mobile phone, a smartphone, a tablet, a personal computer, a workstation, a server, an edge computing device, or other electronic device. The one or more computing devices 105 may be enabled with Internet or other network access and may be locatable by the at least one network 110. In one aspect, the one or more computing devices 105 may be configured to transmit instructions to control various devices, receive data or other signals from external devices, and transmit the received data or other data to the at least one network 110, a server, or another remote computing device 105 of the network environment 100.

In the example of FIG. 1, the computing device 105-a may include a non-contact sensor 130 and a MMRT system 140 including an I/O component 142 and a data analyzation component 144. The I/O component 142 may handle input and output operations for the MMRT system 140, including receiving respiratory motion data from the non-contact sensor 130 and transmitting control signals to a music source. For example, the I/O component 142 may receive real-time magnetic flux data reflecting the user's inhalation and exhalation cycles and may transmit feedback signals to select, modulate, or generate music based on one or more music characteristics. The data analyzation component 144 may process and analyze received respiratory motion data, extract respiratory features, and generate feedback signals for controlling the music output. In certain examples, the data analyzation component 144 may extract features including breathing rate derived from peak-to-peak interval (PPI) values, breathing uniformity expressed as the coefficient of variation (CV) of PPI values, breathing amplitude, and spectral features identified via Fast Fourier Transform (FFT) analysis. For example, the data analyzation component 144 may determine that a user's breathing rate has increased from a baseline of 15 breaths per minute to 21 breaths per minute during exposure to a particular music selection, and may generate a feedback signal to select, modulate, or generate music to guide the user's breathing rate toward a target condition. In certain examples, the MMRT system 140 may also be accessible to users via the user devices 105 through the communication links 120 and the cloud infrastructure 110 as described herein.

Additionally, in some cases, the non-contact sensor 130 may be configured to detect respiratory motion of a user 134 without requiring physical attachment to the user's body. In certain examples, the non-contact sensor 130 may operate on the basis of magnetic sensing: a small permanent magnet may be placed on the user's chest or clothing, and movement of the magnet caused by breathing may alter the magnet's position relative to a fixed magnetic sensor (including, for example, a Hall sensor) positioned nearby. The Hall sensor may operate based on the Hall effect, which occurs when a magnetic field is applied perpendicular to the flow of electric current in a conductor, producing a Hall voltage proportional to the strength of the magnetic field. As the user inhales, the permanent magnet may move away from the Hall sensor, resulting in a lower detected magnetic flux, and as the user exhales, the permanent magnet may move closer to the Hall sensor, resulting in a higher detected magnetic flux. The resulting time-varying magnetic flux signal may produce a real-time waveform reflecting the user's respiratory motion, from which respiratory features such as breathing rate, breathing amplitude, and breathing regularity may be derived. For example, a user breathing at a rate of 18 breaths per minute may produce a periodic magnetic flux signal with approximately 18 peaks per minute, and variations in the amplitude of those peaks may indicate irregularity in the user's breathing depth. In various examples, the non-contact sensor 130 may communicate with the MMRT system 140 via a wireless signal 132.

In various examples, the MMRT system 140 may leverage the observed phenomenon that breathing tends to synchronize with the rhythm and tempo of music following initial exposure. For example, when a user listens to music, the user's breathing may synchronize with the rhythm and tempo of the music within approximately ten to twenty-five seconds. The MMRT system 140 may exploit this synchronization by selecting, modulating, or generating music based on one or more music characteristics to modulate the user's detected respiratory patterns toward a target condition. As used herein, music characteristics may include, without limitation, tempo, volume, rhythm, pitch, melodic phrasing, harmonic content, dynamic variation, timbre, rhythmic complexity, tonal mode, genre, or combinations thereof. In various examples, the MMRT system 140 may select music from a music library, modulate one or more parameters of a current music output, generate music algorithmically or via one or more machine learning models, or employ any combination thereof, based on the user's detected respiratory patterns. For example, if the non-contact sensor 130 detects that a user's breathing rate is elevated relative to a target condition, the MMRT system 140 may select or generate music having a slower tempo to guide the user's breathing rate downward. Conversely, if the user's breathing rate is below a target condition, the MMRT system 140 may select or generate music having a faster tempo to encourage a higher breathing rate. In certain examples, soothing piano music may be selected to promote relaxation and reduce breathing rate in a user exhibiting elevated respiratory rates, while a different music selection may be used to increase alertness or breathing depth as appropriate for the user's condition.

The one or more computing devices 105 may interface with one or more data stores accessible via the cloud infrastructure 110 or locally within the computing environment. The one or more data stores may persist information related to the operation of the MMRT system 140, including, for example, respiratory motion data collected from users, extracted respiratory features such as breathing rate, breathing uniformity, and spectral features, reference respiratory patterns organized by demographic group (including, for example, subjects under 19 years of age, subjects between 19 and 59 years of age, and subjects over 60 years of age), machine learning model parameters, music library metadata, and longitudinal respiratory health profiles for individual users. For example, the one or more data stores may persist baseline respiratory data for a user collected during an initial session, enabling the MMRT system 140 to compare subsequent sessions against the baseline to detect changes in the user's respiratory health over time. Furthermore, the one or more data stores may persist population-level respiratory data collected from a plurality of subjects under various conditions, including normal breathing without music, breathing while listening to soothing piano music, and breathing while listening to hard rock music, to support the training and refinement of machine learning models used by the MMRT system 140.

The various types of data may be maintained in a single data store or distributed across multiple data stores, including, for example, relational databases, document stores, object storage services, data lakes, or combinations thereof. In certain examples, data may be stored in a centralized repository accessible to the computing devices 105 and the MMRT system via the network 110 or the cloud infrastructure 110. In other examples, data may be distributed across multiple nodes or regions using distributed storage architectures, enabling scalable access and retrieval with consistency guarantees appropriate to the application.

In some examples, the one or more networks 110 may be at least one local area network (LAN), at least one wide area network (WAN), or other types of networks, supporting protocols like transmission control protocol/Internet protocol (TCP/IP) over Ethernet. In some instances, the one or more networks 110 may be a cloud network. The one or more communication networks 110 may serve as a system hub, providing computational resources and storage capabilities. The one or more communication networks 110 may include resources to manage and deliver computing services over the Internet or other communication forms. The one or more communication networks 110 may be executed within or as a virtual machine or a virtual server that may be managed in a cloud-based computing environment. Also, the application(s) may be located on one or more virtual server(s) running in a cloud-based computing environment rather than being tied to one or more specific physical network computing devices. For example, the application(s) may be running in one or more virtual machines (VMs). In some examples, the one or more networks 110 may include elements such as data centers, virtualized resources, cloud services, and security tools. In some aspects, the one or more communication networks 110 may utilize data centers that also host servers capable of processing, storing, and managing network traffic.

In some examples, the one or more networks 110 may include one or more application servers coupled to a plurality of service instances that may host a plurality of data stores and serve a plurality of client or user devices via the network 110. The one or more application servers may store one or more applications that can include executable instructions that, when executed by the one or more application servers, cause the one or more application servers to perform desired actions, such as to transmit, receive, or otherwise process network messages, for example, and to perform other actions described and illustrated herein with reference to the figures. The one or more application servers may work alongside the MMRT system 140 for monitoring respiratory motion of a user in real time and providing adaptive music-based feedback designed to modulate the user's breathing. The applications may be implemented as software modules or components of other applications. Further, the applications can be implemented as operating system extensions, components, plugins, or the like.

Additionally, in some examples, elements within the network environment 100 may include components such as processors, memory, displays, input devices, network interfaces, storage devices, and output devices. The network environment 100 may provide computational and storage capabilities, including processing power, memory, networking, and persistent storage, required for the operation of one or more computing programs. In some instances, the network environment 100 may integrate cloud computing, where applications may access computing resources on demand, enabling the scaling of resources based on computational and storage requirements. The processors may be configured to execute software instructions for performing functions described herein, in addition to other functions such as receiving queries, analyzing the queries, retrieving resolutions, and displaying results.

The techniques described herein provide systems and methods for monitoring respiratory motion of a user in real time and providing adaptive music-based feedback designed to modulate the user's breathing toward an optimal or target condition. For example, during sleep, the MMRT system 140 may continuously monitor a user's breathing patterns and detect abnormal variations indicative of an approaching apnea event, such as a progressive decrease in breathing amplitude or an irregular breathing rhythm. In response, the MMRT system 140 may adjust the music output to restore optimal breathing conditions, for example, by modifying the tempo or rhythm of the music to encourage more regular and deeper breathing. In another example, during respiratory rehabilitation for a user recovering from COVID-19 or other respiratory conditions, the MMRT system 140 may select music parameters tailored to the user's demographic group and health status to promote controlled breathing exercises and relaxation. If the MMRT system 140 determines that music parameter adjustments are insufficient to restore the user's breathing to an optimal condition, the MMRT system 140 may generate an alert, including, for example, transmitting a notification to a designated contact or healthcare provider via a network-connected device.

In various examples, the MMRT system 140 may support additional therapeutic applications beyond sleep monitoring and respiratory rehabilitation. For example, music with a steady rhythm may assist users in practicing paced breathing techniques, in which the user synchronizes breath with the tempo of the music to achieve more controlled and deeper breathing beneficial for respiratory therapy. The MMRT system 140 may select or generate music having a tempo corresponding to a target breathing rate, enabling the user to entrain breathing to the music tempo and thereby practice paced breathing without requiring active clinical supervision. In certain examples, the MMRT system 140 may also be used to alleviate anxiety and stress, as calming music may activate the parasympathetic nervous system, promoting relaxation and slowing the respiratory rate. High stress may lead to rapid, shallow breathing or difficulty in regulating breath, and the MMRT system 140 may counteract such effects by providing music selected or generated to promote relaxation and respiratory stability. In further examples, the MMRT system 140 may support pain management applications, as music therapy may indirectly enhance breathing patterns by reducing physical and emotional stress associated with respiratory symptoms or other conditions. By alleviating pain and discomfort through music-based intervention, the MMRT system 140 may promote more regular and stable breathing patterns, and improved emotional well-being may contribute to greater motivation and engagement in respiratory therapies. Additionally, the MMRT system 140 may enhance a user's awareness of breathing patterns, encouraging slower and more deliberate breathing, which may improve respiratory control and symptom management across a variety of clinical and wellness contexts.

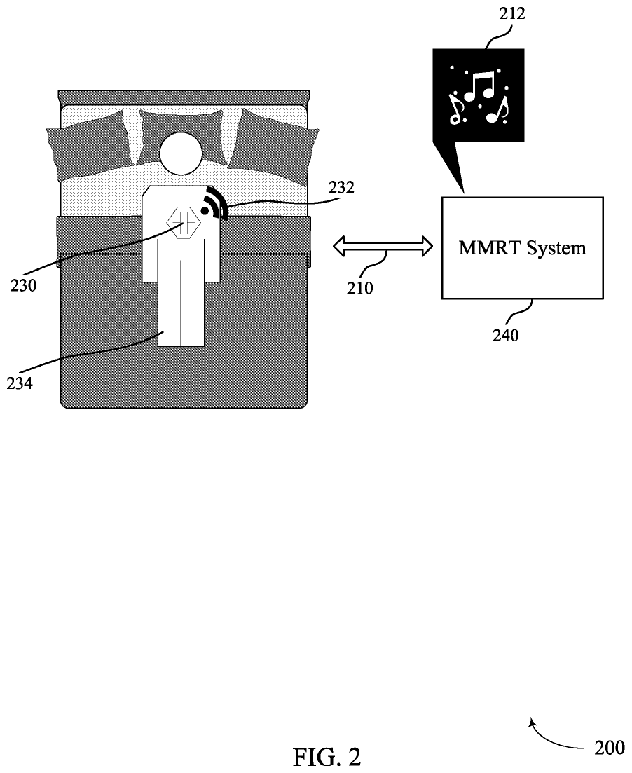

FIG. 2 illustrates an example system 200 in which an MMRT system 240 is deployed in a bedroom or sleep environment according to some examples. In this example, a user 234 may be lying in bed, and a non-contact sensor 230 may monitor the user's breathing via wireless signal 232 without requiring the user to wear any attached sensing device on the user's 234 skin. In certain examples, the non-contact sensor 230 may comprise a small permanent magnet affixed to the user's chest or clothing and a fixed magnetic sensor, including, for example, a Hall sensor, positioned at a bedside surface or integrated into a bedside device at a predetermined distance from the magnet. As the user 234 inhales, the permanent magnet may move away from the Hall sensor, resulting in a lower detected magnetic flux, and as the user 234 exhales, the permanent magnet may move closer to the Hall sensor, resulting in a higher detected magnetic flux, thereby producing a time-varying magnetic flux signal that reflects the user's respiratory motion in real time. Music output 212 may be provided to the user, for example, via speakers, a bedside device, or headphones. In certain examples, the music volume may be maintained at a level that is comfortable and safe for extended listening without risking hearing damage, including, for example, approximately 65 decibels. The MMRT system 240 may receive respiratory motion data from the non-contact sensor 230, extract respiratory features including, for example, breathing rate derived from peak-to-peak interval (PPI) values, breathing uniformity expressed as the coefficient of variation (CV) of PPI values, breathing amplitude, and spectral features identified via Fast Fourier Transform (FFT) analysis, and analyze the extracted features to determine whether the user's current breathing pattern falls within an optimal or target range. Based on this analysis, the MMRT system 240 may select, modulate, or generate music based on one or more music characteristics to adjust the music output 212 in real time. In various examples, the MMRT system 240 may leverage the music-breathing synchronization phenomenon described herein, such that music output having particular characteristics may be used to shift the user's breathing rate or depth in a desired direction. For example, soothing piano music may be selected to promote relaxation and reduce breathing rate, as piano music has been observed to elicit relaxation and enhance breathing amplitude regularity, while hard rock music has been observed to increase breathing rate and introduce irregularity in breathing amplitude in certain subjects. This sleep-environment configuration may be used for monitoring and managing respiratory disturbances during sleep, including, for example, sleep apnea, irregular breathing amplitude, and breathing pattern changes associated with dream states or sleep disorders. In certain examples, the MMRT system 240 may detect abnormal variations in the user's breathing pattern indicative of an approaching apnea event, such as a progressive decrease in breathing amplitude or an irregular breathing rhythm, and may select, modulate, or generate music to restore optimal breathing conditions. For users recovering from COVID-19 or other respiratory conditions, the MMRT system 240 may provide music tailored to the user's demographic group and health status to promote controlled breathing exercises and relaxation during sleep. For users with disrupted sleep patterns, the MMRT system 240 may provide calming music to stabilize breathing and improve sleep quality, thereby supporting overall recovery and respiratory health.

As shown, the system 200 may include the non-contact sensor 230 in communication with the MMRT system 240 via the wireless signal 232. The wireless signal 232 may support bidirectional data flow, enabling the non-contact sensor 230 to transmit real-time magnetic flux data reflecting the user's inhalation and exhalation cycles to the MMRT system 240, and enabling the MMRT system 240 to transmit control signals or configuration parameters back to the non-contact sensor 230. In certain examples, the system 200 may further include at least one communication link 210 supporting communication between the MMRT system 240 and other components of the system 200. The MMRT system 240 may process the received respiratory motion data, generate a feedback signal specifying a music selection, one or more parameter modulations, music generation instructions, or a combination thereof, and transmit the feedback signal to a music source to control the music output 212 in real time, thereby forming a closed-loop therapeutic system in which the user's respiratory state is continuously monitored and the music output is dynamically adapted to modulate the user's breathing toward an optimal or target condition. In certain examples, if the MMRT system 240 determines that the music output is insufficient to restore the user's breathing to an optimal condition, or if an anomalous breathing pattern meets or exceeds a specified threshold, the MMRT system 240 may generate an alert, including, for example, transmitting a notification to a designated contact or healthcare provider via a network-connected device.

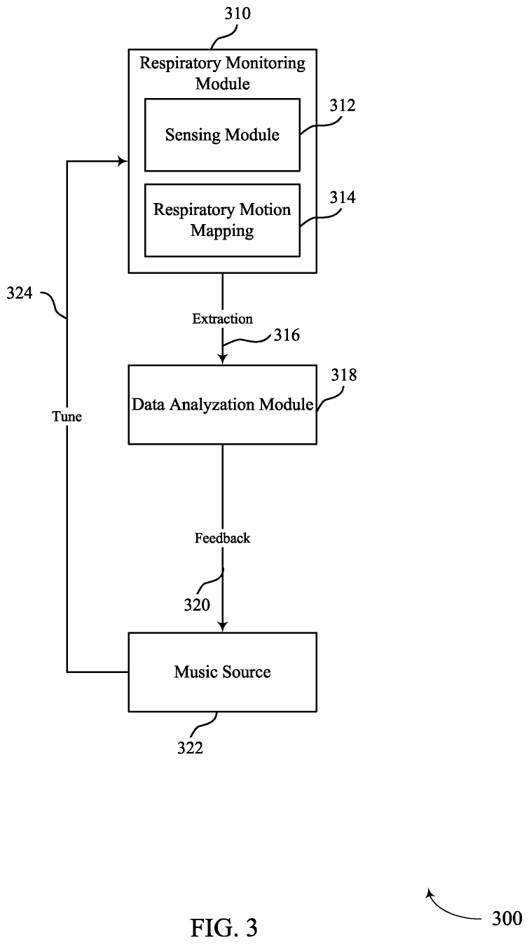

FIG. 3 illustrates an example system flow 300 for the MMRT system according to some examples. Here, FIG. 3 illustrates how the data flow and processing steps by which respiratory motion is monitored and music is adjusted according to some examples. The system flow 300 may include a respiratory monitoring component 310, a data analyzation component 318, a music source 322, and associated feedback components. The system flow 300 may be particularly suited for monitoring respiratory signals during periods of rest, when the medulla oblongata, situated within the brainstem, assumes primary responsibility for regulating the respiratory rhythm. The medulla oblongata may receive information from chemoreceptors that detect chemical fluctuations in the bloodstream, including levels of oxygen and carbon dioxide, and may orchestrate responses within the nervous system to modulate respiratory activity. Because the medulla also governs the sympathetic nervous system, which operates autonomously and independently of conscious control, respiratory signals captured during rest may provide insights into an individual's physiological state that remain beyond conscious manipulation. The system flow 300 may leverage this characteristic by monitoring the user's respiratory patterns in real time and providing adaptive music-based feedback to modulate breathing through the medulla's response to auditory stimuli.

In various examples, the system flow 300 may further account for the neural mechanisms by which music influences respiratory function. The temporal lobe, which is responsible for auditory perception, may process musical stimuli and relay information to the amygdala, a center for emotional processing that responds to the emotional cues conveyed by music. The interaction between these brain regions and the medulla oblongata may modulate respiratory activity, such that music perceived as calming or soothing may activate the parasympathetic nervous system, which promotes relaxation and slows the respiratory rate. Conversely, music perceived as stimulating or arousing may elicit sympathetic nervous system responses, resulting in elevated respiratory rates and reduced breathing regularity. In certain examples, music may affect breathing partly through emotional mediation, as different types of music may evoke emotional responses that trigger physiological changes, including alterations in heart rate and respiratory rate. The system flow 300 may leverage these neural and physiological pathways by selecting, modulating, or generating music based on one or more music characteristics to elicit a desired parasympathetic or sympathetic response, thereby modulating the user's respiratory patterns toward a target condition.

The respiratory monitoring component 310 may be responsible for capturing and tracking the user's breathing patterns in real time. The respiratory monitoring component 310 may include a sensing component 312, a respiratory motion mapping component 314, followed by an extraction 316 step leading to the next portion of the system flow 300. The sensing component 312 may perform the detection of respiratory motion, for example, by receiving magnetic flux data from a non-contact sensor, including, for example, the non-contact sensor 130 of FIG. 1. The respiratory motion mapping component 314 may convert raw sensor data into a representation of the user's breathing pattern over time, such as a time-series signal of magnetic flux values corresponding to inhalation and exhalation cycles. In certain examples, the respiratory motion mapping component 314 may segment the time-series signal into predetermined intervals, including, for example, twenty-second intervals, to facilitate examination of the temporal progression of respiratory behavior over the duration of a monitoring session. This temporal segmentation may enable the system flow 300 to capture how the user's respiratory patterns evolve over time in response to music exposure, rather than relying on a single aggregate measurement. The extraction 316 may isolate relevant respiratory features from the mapped respiratory motion data. Respiratory features that may be extracted include, for example, breathing rate (derived from peak-to-peak interval (PPI) values, where the breathing rate may be calculated as sixty divided by the mean PPI value), breathing uniformity (expressed as the coefficient of variation (CV) of PPI values, calculated as the standard deviation of PPI values divided by the mean PPI value), breathing amplitude, and spectral features (including, for example, dominant frequency components identified via Fast Fourier Transform (FFT) analysis, which may translate the respiratory signal between the time and frequency domains to identify dominant frequencies and characterize the regularity and variability of breath cycles).

In certain examples, the FFT analysis described above may also serve as a diagnostic tool for identifying abnormal breathing patterns. FFT may effectively identify unusual respiratory patterns, including periods of apnea and irregular rhythms, which may indicate respiratory issues or other health conditions. For example, spectral analysis of a healthy subject's respiratory signal may reveal a single dominant frequency peak corresponding to a regular breathing rate, whereas spectral analysis of a subject with a respiratory condition, including, for example, COVID-19, may reveal multiple frequency components, broadened spectral peaks, or shifted dominant frequencies indicative of irregular or compromised respiratory function. The contrast in spectral characteristics between healthy and diseased breathing patterns may enable the data analyzation component 318 to classify a user's respiratory state and to detect respiratory anomalies that may not be readily apparent from time-domain analysis alone.

The data analyzation component 318 may receive the extracted respiratory features from extraction 316 and interpret those features to determine appropriate music adjustments. In certain examples, the data analyzation component 318 may utilize normal breathing patterns as a baseline and compare deviations in respiratory patterns induced by music exposure against the baseline. The data analyzation component 318 may compare the extracted features against target or reference values to determine whether the user's current breathing pattern falls within an optimal or target range. In certain examples, the comparison may reveal that the impact of music on respiratory patterns is not static but dynamic, evolving over time as the user is exposed to music. For example, deviations in breathing rate and coefficient of variation may exhibit fluctuating trends over the course of a monitoring session, transitioning between positive and negative deviations before settling into a more stable trajectory. The data analyzation component 318 may account for these temporal dynamics when generating music adjustment recommendations. In certain examples, the data analyzation component 318 may also account for individual variability in music response, as different users may exhibit neutral, positive, or negative respiratory responses to the same music. For example, a first user may exhibit a reduced breathing rate and enhanced breathing amplitude regularity in response to piano music, indicating a positive relaxation effect, while a second user may exhibit an increased breathing rate and irregular breathing amplitude in response to the same music, indicating a negative effect on breathing performance. A third user may exhibit no significant change in breathing rate but may show improved or degraded breathing regularity. Based on this comparison, the data analyzation component 318 may generate a feedback signal 320 to the music source 322, specifying adjustments to one or more music parameters. These parameters may include, for example, tempo, volume, rhythm, pitch, and music selection or genre. In certain examples, the steady rhythm of piano music may be selected to help regulate breath patterns, as listeners may unconsciously synchronize their breathing to the music's tempo, promoting a more even and controlled breathing rate, while music perceived as soothing or uplifting may be more likely to yield beneficial effects on breathing and overall well-being. The music source 322 may comprise an audio library, streaming service, or other audio output component, and may adjust and tune 324 in response to the feedback signal 320. In certain examples, the user's breathing and the music may become synchronized (coupled) after the user has been exposed to the music for a period of time. For example, the period of time may be approximately ten to twenty-five seconds, at which point adjustments to music parameters may be used to shift the user's breathing rate or depth in a desired direction. In certain examples, the data analyzation component 318 may continue to monitor the user's respiratory features after a music adjustment has been applied, and may generate subsequent feedback signals 320 to iteratively refine the music output until the user's breathing pattern converges toward the target or optimal condition.

For example, in experimental testing, a first subject exhibited a breathing rate of approximately twenty-two breaths per minute under normal conditions, approximately twenty-one breaths per minute while listening to piano music, and approximately eighteen breaths per minute while listening to rock music. Although rock music reduced the first subject's breathing rate, the irregularity in breathing amplitude associated with rock music indicated a negative impact on the first subject's overall breathing performance. A second subject exhibited a breathing rate of approximately twenty-one breaths per minute under normal conditions, approximately twelve breaths per minute while listening to piano music, and approximately twenty-one breaths per minute while listening to rock music. Piano music dramatically reduced the second subject's breathing rate and enhanced the regularity of breathing amplitude, indicating a strong positive relaxation effect. A third subject exhibited an increased breathing rate in response to both piano and rock music, and the irregularity in breathing amplitude persisted despite the music, indicating a negative impact on the third subject's breathing performance. These illustrative examples demonstrate that the data analyzation component 318 may account for the range of individual variability in respiratory response to music when generating feedback signals 320 to the music source 322, and may adapt music parameter adjustments based on the observed effect of a given music selection on the individual user's respiratory features rather than relying solely on population-level trends.

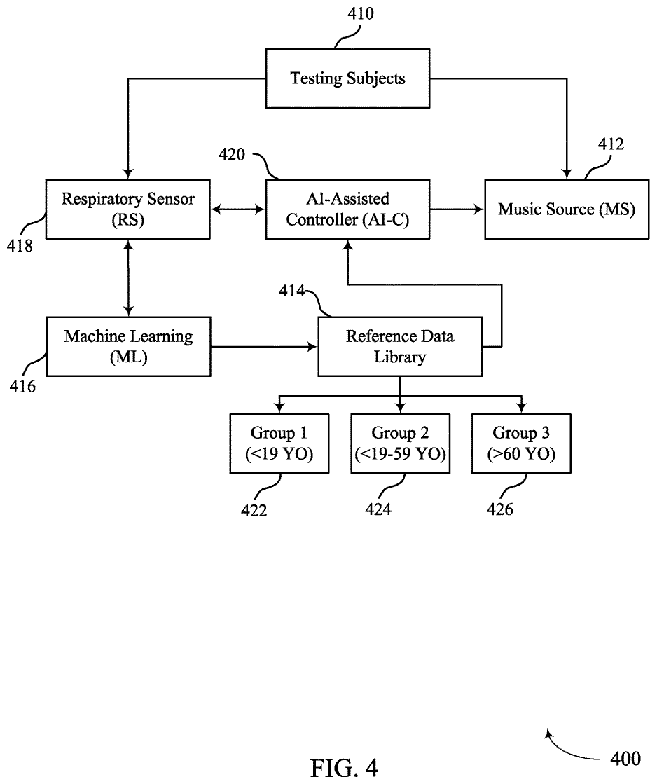

FIG. 4 illustrates an example system schematic 400 for an AI-assisted MMRT system according to some examples. The system schematic 400 may include testing subjects 410, a music source (MS) 412, a reference data library 414, a machine learning (ML) component 416, a respiratory sensor (RS) 418, and an AI-assisted controller (AI-C) 420.

Here, testing subjects 410 may include human participants from whom respiratory data is collected for training and validating the machine learning component 416. In various examples, the testing subjects 410 may be organized into demographic groups, including a first group, such as Group I 422, comprising subjects under 19 years of age, a second group, Group II 424, comprising subjects between 19 and 59 years of age, and a third group, Group III 426, comprising subjects over 60 years of age. Additional groupings based on health status, including, for example, healthy subjects, COVID-19 patients, lung disease patients, and heart disease patients, may also be used. The age-based and condition-based grouping may reflect the observation that music influences respiratory patterns differently across age groups and health conditions. In experimental testing, ninety-seven subjects participated in a study adhering to protocols involving breathing without music to establish a normal baseline, breathing while listening to soothing piano music, and breathing during exposure to hard rock music, with each respiratory measurement spanning approximately three minutes. The subjects were classified into the three demographic groups described above. Subjects in Group I 422 exhibited the greatest variation in respiratory response when transitioning between different music styles, indicating that younger subjects were most sensitive to changes in music genre. Subjects in Group II 424 exhibited a stabilization of breathing patterns in response to music exposure, indicating that music positively influenced and regulated respiratory function in middle-aged subjects. Subjects in Group III 426 processed musical stimuli differently than younger groups, and the impact of music on respiratory patterns diminished as age increased, with the oldest group exhibiting notable irregularity in respiratory deviation values and responses that remained closer to baseline compared to the other groups. Additionally, two volunteer patients with COVID-19 were exposed to music following the same testing protocol. A first COVID-19 patient exhibited a normal breathing rate of approximately fifteen breaths per minute without music, approximately sixteen breaths per minute while listening to piano music, and approximately eighteen breaths per minute while listening to rock music. While piano music slightly increased the breathing rate, it also enhanced the regularity of breathing amplitude, indicating a positive impact on the patient's breathing performance. A second COVID-19 patient exhibited a normal breathing rate of approximately fourteen breaths per minute without music, approximately twelve breaths per minute while listening to piano music, and approximately fourteen breaths per minute while listening to rock music. In addition to achieving a reduced breathing rate, piano music facilitated relaxation and promoted more regular breathing in the second patient.

The respiratory sensor (RS) 418 may capture breathing data from subjects or users. In certain examples, the respiratory sensor 418 may comprise a non-contact magnetic sensor that detects respiratory motion via changes in magnetic flux from a permanent magnet placed on the subject's or user's chest or clothing. In other examples, the respiratory sensor 418 may comprise any other suitable sensor capable of detecting respiratory motion with sufficient sensitivity to capture changes in breathing patterns, including, for example, Hall sensors, other magnetic sensors, or other suitable sensing technologies.

The reference data library 414 may be a structured database containing respiratory patterns and associated metadata organized by demographic group, including, for example, age group and health status. In various examples, the reference data library 414 may include breathing rate data, breathing uniformity data, such as CV of PPI values, and FFT spectral data collected from subjects in each demographic group under various conditions. This may include normal breathing without music, breathing while listening to soft piano music, and breathing while listening to hard rock music. In certain examples, the reference data library 414 may further include deviation data representing the difference between normal breathing patterns and music-induced respiratory patterns for each subject, organized by temporal interval. For example, the reference data library 414 may store, for each subject, the deviation in breathing rate and coefficient of variation between normal breathing and piano-induced breathing, and between normal breathing and rock-induced breathing, computed for each predetermined temporal interval, including, for example, each twenty-second interval over a three-minute measurement window. The reference data library 414 may further include positive percentage values indicating the proportion of subjects for whom a given music condition produced a positive effect on breathing rate or breathing regularity relative to normal breathing within each temporal interval. In experimental testing, during a final temporal interval of a three-minute monitoring session, approximately seventy-three percent of subjects exhibited a reduced breathing rate and increased breathing regularity during piano music exposure relative to normal breathing, signifying a positive respiratory outcome for the majority of subjects. In contrast, rock music exposure produced predominantly negative deviations in average breathing rate across most temporal intervals, with positive percentages remaining below fifty percent, indicating that rock music induced elevated respiratory rates and compromised breathing regularity for the majority of subjects tested. The reference data library 414 may also store age-group-specific deviation data, enabling the AI-assisted controller 420 and the machine learning component 416 to identify age-dependent patterns in music response and to tailor music therapy accordingly. The reference data library 414 may be stored in a database accessible to the AI-assisted controller 420 and the machine learning component 416.

Further, the machine learning (ML) component 416 may analyze respiratory data from the reference data library 414 and from real-time user sessions to learn associations between respiratory features and subject characteristics to generate predictions about a user's respiratory state and appropriate music parameters. In various examples, the machine learning component 416 may employ supervised learning algorithms trained on labeled respiratory data, where labels may include, for example, demographic group, health condition, and music condition. In prior experimental validation, the machine learning component 416 achieved a classification accuracy of over ninety percent in identifying respiratory anomalies, including distinguishing between breathing patterns of healthy individuals and patients with respiratory conditions such as COVID-19. In certain examples, data acquired during each user session may be used to refine the machine learning models and to build a longitudinal respiratory health profile for the individual user. The machine learning component 416 may be capable of differentiating between breathing patterns associated with different conditions, including, for example, healthy breathing, COVID-19-associated breathing anomalies, apnea precursor events, lung disease patterns, and heart disease patterns. In various examples, the machine learning component 416 may analyze breathing pattern data classified into the three age groups, Group I 422, Group II 424, and Group III 426, to inform the AI-assisted controller 420, enabling the AI-assisted controller 420 to quickly and accurately determine optimal breathing conditions for an unknown subject based on provided information such as age, gender, and preferred music. For example, the machine learning component 416 may determine that a subject in Group II 424 is likely to respond positively to piano music based on population-level data indicating that middle-aged subjects generally exhibit stabilized breathing patterns during piano music exposure, and may recommend piano music as an initial music selection for that subject. Machine learning models and algorithms that may be used include, for example, classification algorithms, regression algorithms, anomaly detection algorithms, clustering algorithms, or other suitable machine learning approaches, or combinations thereof.

The AI-assisted controller (AI-C) 420 may manage both the respiratory sensor 418 and the music source 412, serving as the central control component of the AI-MMRT system. In various examples, the AI-assisted controller 420 may receive real-time respiratory motion data from the respiratory sensor 418 and invoke the machine learning component 416 to analyze the data and classify the user's respiratory state. Based on the user's demographic information and health status, the AI-assisted controller 420 may determine an optimal or target breathing condition for the user and generate a feedback signal specifying adjustments to one or more music parameters of the music source 412, which may then be transmitted to the music source 412 to adjust the music in real time. In certain examples, the AI-assisted controller 420 may also detect anomalous breathing patterns (including, for example, patterns indicative of an approaching apnea event or of a respiratory disturbance) before or as the disturbance occurs, and may respond by adjusting music parameters to attempt to restore the user's breathing to an optimal condition. In certain examples, if music parameter adjustment does not successfully restore optimal breathing conditions, or if the anomaly meets or exceeds a specified threshold, the AI-assisted controller 420 may generate an alert, such as by transmitting a notification to the user's designated contacts, including, for example, family members, friends, or healthcare providers via a network-connected device.

The music source (MS) 412 may be any audio library, streaming service, or adaptive audio output component capable of providing music to the user and adjusting music parameters in response to control signals from the AI-assisted controller 420. In certain examples, a smartphone may serve as both the music source and a control interface, receiving respiratory data from a sensing device via a wireless link and processing the data through software that links the sensing data to the music output. In various examples, the user may listen to music via a headset connected to the smartphone, and the music volume may be maintained at a level that is comfortable and safe for extended listening without risking hearing damage, including, for example, approximately sixty-five decibels. Music parameters that may be adjusted by the AI-assisted controller 420 include, for example, tempo, volume, rhythm, pitch, and music selection or genre. In certain examples, increasing music tempo may be used to increase the user's breathing rate, and decreasing music tempo may be used to decrease it, leveraging the music-breathing synchronization phenomenon described herein. In various examples, engaging with music may enhance the user's mindfulness of breathing, encouraging the user to focus on breath and become more aware of breathing patterns, potentially leading to more intentional and regulated respiration. A clear understanding of music's effects on individual breathing patterns may allow for the customization of music selections to enhance respiratory performance, supporting applications in medical treatment, respiratory therapy, and health exercises.

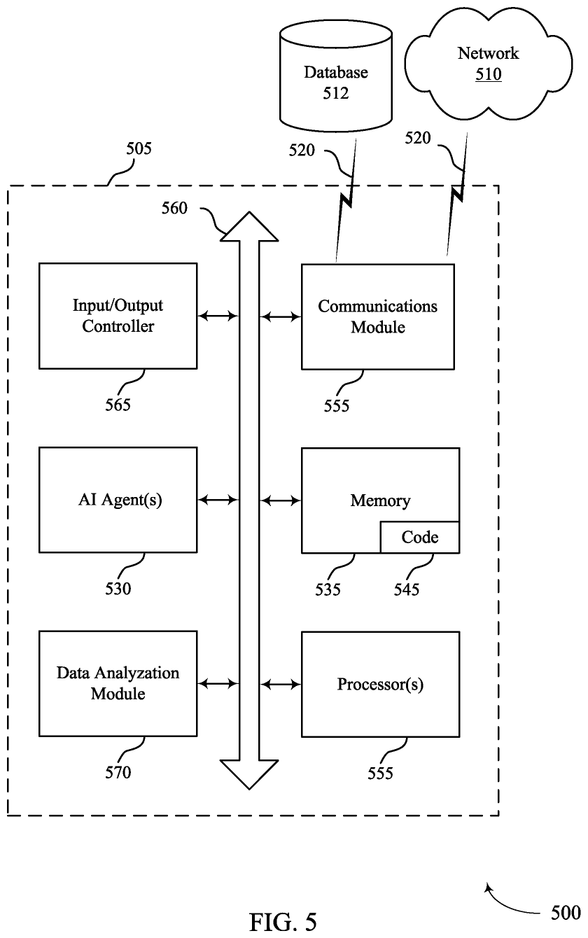

FIG. 5 illustrates an example computing environment 500 for an MMRT system 505 according to some examples, providing a more detailed view of the hardware and software architecture that may implement the MMRT system. The computing environment 500 may include a network 510, a database 512, one or more network connections 520, one or more AI agents 530, memory 535 having processor-accessible code 545, a communications component 555, an input/output controller 565, a data analyzation component 570, and one or more processors 575.

The network 510 may be any suitable communication infrastructure connecting the MMRT system 505 to external resources, including, for example, local area networks, wide area networks, the Internet, private networks, or combinations thereof. The database 512 may serve as external data storage for respiratory patterns, music libraries, reference data, and machine learning model parameters, accessible to the MMRT system 505 via the network connections 520. The network connections 520 may be interfaces enabling communication between the MMRT system 505 and network resources, and may utilize any of the communication protocols or technologies described with respect to the communication links 120 of FIG. 1, or other suitable protocols.

Memory 535 may be any suitable volatile or non-volatile storage, including, for example, random access memory (RAM), read-only memory (ROM), flash memory, solid-state drives (SSDs), or combinations thereof. The memory 535 may store code 545 comprising software instructions which, when executed by one or more processors 575, cause the MMRT system 505 to perform the functions described herein.

The communications component 555 may manage network protocols and data transmission between the MMRT system 505 and external devices, including, for example, the non-contact sensor 130, the user devices 105, and the cloud infrastructure 110.

The AI agents 530 may be intelligent software components that make decisions about music modulation, respiratory state classification, and anomaly detection within the MMRT system 505. In certain examples, the AI agents 530 may correspond to or implement the machine learning component 416 described with respect to FIG. 4. Multiple AI agents 530 may operate concurrently, with different agents handling different aspects of the system's decision-making, including, for example, real-time music parameter adjustment and longitudinal health trend analysis.

The input/output controller 565 may be a hardware interface managing connections to sensors, including, for example, the non-contact sensor 130 or other respiratory sensing devices, and output devices, including, for example, speakers or displays communicating music or other output to the user.

The data analyzation component 570 may be a software component that processes respiratory data received from sensors, extracts respiratory features, interfaces with the AI agents 530 to classify the user's respiratory state and determine appropriate music adjustments, and generates feedback signals to the music source. In certain examples, the data analyzation component 570 may correspond to or implement the data analyzation component 144 of FIG. 1 and the data analyzation component 318 of FIG. 3.

The processors 575 may be any suitable hardware processor or combination of processors, including, for example, a central processing unit (CPU), a graphics processing unit (GPU), a neural processing unit (NPU), an application-specific integrated circuit (ASIC), a field-programmable gate array (FPGA), or combinations thereof.

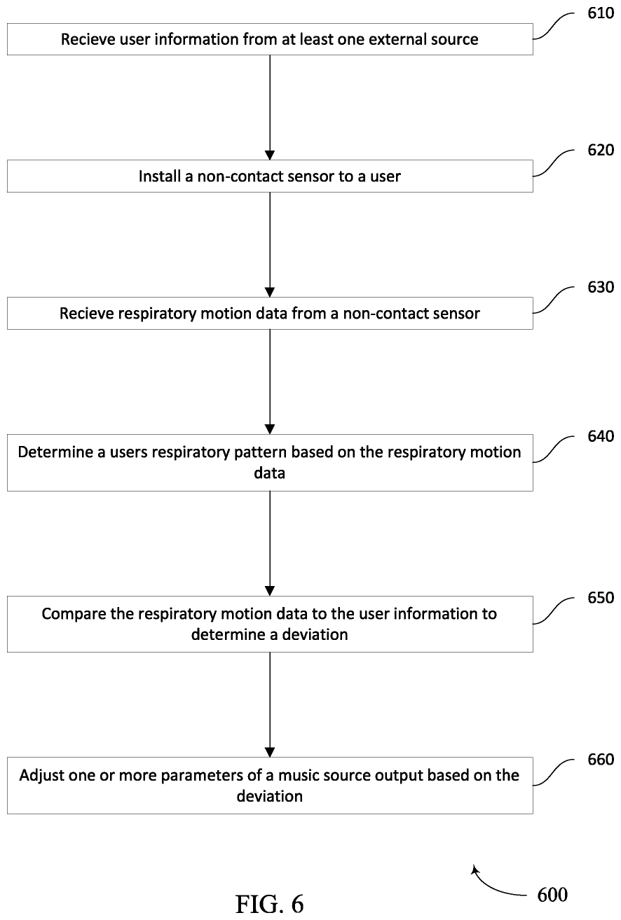

FIG. 6 illustrates an example method flow for operating the MMRT system according to some examples. FIG. 6 is a flowchart of an example method for music-modulated respiratory therapy, in which a system receives user information, installs and activates a non-contact sensor to monitor a user's respiratory motion, determines the user's respiratory pattern from the acquired data, compares the respiratory pattern data against the user information to identify deviations from a target or optimal condition, and adjusts one or more parameters of a music source output based on the identified deviation. The method steps described below may be performed by one or more components of the MMRT system, including, for example, the AI-assisted controller 420 of FIG. 4, the data analyzation component 318 of FIG. 3, or the data analyzation component 570 of FIG. 5, individually or in combination.

At 610, the method may include receiving user information from at least one external source. The user information may include demographic data such as the user's age, health status, medical history, and any pre-existing conditions affecting the cardiorespiratory system, including, for example, lung disease, heart disease, post-COVID-19 respiratory complications, or sleep apnea. In certain examples, the at least one external source may include electronic health records, user-provided input via a user device, data from the reference data library 414, or other clinical or personal data sources accessible via a network. The received user information may be used by the AI-assisted controller 420 or the machine learning component 416 to determine an appropriate demographic group classification for the user, such as Group I 422, Group II 424, or Group III 426, as described with respect to FIG. 4, and to establish a target or optimal respiratory condition against which the user's real-time respiratory data may be compared.

At 620, the method may include installing a non-contact sensor on a user. In certain examples, installing the non-contact sensor may comprise positioning a small permanent magnet on the user's chest or clothing and placing a fixed magnetic sensor, including, for example, a Hall sensor, at a predetermined distance from the magnet. The non-contact sensor, including, for example, the non-contact sensor 130 of FIG. 1 or the non-contact sensor 230 of FIG. 2, may be configured to detect changes in magnetic flux caused by movement of the permanent magnet as the user breathes, without requiring physical attachment of the sensor to the user's body. In various examples, the non-contact sensor may be positioned in a sleep environment, such as on a bedside surface or integrated into a bedside device, and may communicate with the MMRT system via a wireless signal.

At 630, the method may include receiving respiratory motion data from the non-contact sensor. The non-contact sensor may generate a real-time signal reflecting the user's respiratory motion by detecting changes in magnetic flux produced by the permanent magnet as the user inhales and exhales. In certain examples, the respiratory motion data may be received by the respiratory monitoring component 310, which may include the sensing component 312 for capturing the raw magnetic flux data, the respiratory motion mapping component 314 for converting the raw sensor data into a time-series representation of the user's breathing pattern over time, and the extraction component 316 for isolating relevant respiratory features from the mapped data. Respiratory features that may be extracted include, for example, breathing rate derived from peak-to-peak interval (PPI) values, breathing uniformity expressed as the coefficient of variation (CV) of PPI values, breathing amplitude, and spectral features identified via Fast Fourier Transform (FFT) analysis.

At 640, the method may include determining a user's respiratory pattern based on the respiratory motion data. In certain examples, the data analyzation component, including, for example, the data analyzation component 318 of FIG. 3 or the data analyzation component 570 of FIG. 5, may process the extracted respiratory features to characterize the user's current respiratory state. The determination may include classifying the user's breathing pattern using one or more machine learning models of the machine learning component 416, which may be trained on labeled respiratory data from a plurality of subjects across different demographic groups and health conditions. In various examples, the machine learning component 416 may differentiate between breathing patterns associated with different conditions, including, for example, healthy breathing, COVID-19-associated breathing anomalies, apnea precursor events, lung disease patterns, and heart disease patterns.

At 650, the method may include comparing the respiratory motion data to the user information to determine a deviation. In certain examples, the AI-assisted controller 420 may compare the user's extracted respiratory features against target or reference values to determine whether the user's current breathing pattern falls within an optimal or target range. The target or reference values may be derived from the reference data library 414, which may contain population-level respiratory data organized by demographic group, including, for example, age group and health status. The deviation may represent a difference between the user's current respiratory features and the expected or optimal respiratory features for the user's demographic group and health condition. In certain examples, the comparison may also include detecting anomalous breathing patterns, including, for example, patterns indicative of an approaching apnea event, irregular breathing amplitude, or other respiratory disturbances.

At 660, the method may include adjusting one or more parameters of a music source output based on the deviation. In certain examples, the AI-assisted controller 420 may generate a feedback signal specifying adjustments to one or more music parameters of the music source 412 or the music source 322, and may transmit the feedback signal to the music source to adjust the music output in real time. Music parameters that may be adjusted include, for example, tempo, volume, rhythm, pitch, and music selection or genre. In various examples, increasing the tempo of the music output may be used to increase the user's breathing rate, and decreasing the tempo may be used to decrease the user's breathing rate, leveraging the observed tendency for breathing to synchronize with music tempo after approximately ten to twenty-five seconds of exposure. In certain examples, if the deviation indicates an anomalous breathing pattern that meets or exceeds a specified threshold, and if adjustment of music parameters does not successfully restore the user's breathing to an optimal condition, the AI-assisted controller 420 may generate an alert, including, for example, transmitting a notification to a designated contact or healthcare provider via a network-connected device.

Operations may be performed in the order illustrated, or in any other suitable order consistent with the disclosure. In certain examples, the method may comprise detecting, by a non-contact sensor (including, for example, the non-contact sensor 130 of FIG. 1 or the non-contact sensor 230 of FIG. 2), respiratory motion of a user in real time. The non-contact sensor may detect respiratory motion by monitoring changes in magnetic flux produced by a permanent magnet placed on the user's chest or clothing, without requiring physical attachment to the user's body.

In certain examples, the method may further comprise mapping the detected respiratory motion to a time-series representation of the user's breathing pattern, including, for example, by the respiratory motion mapping component 314 of FIG. 3, and extracting respiratory features from the mapped data, including, for example, by the extraction component 316 of FIG. 3. Respiratory features that may be extracted include, for example, breathing rate, peak-to-peak interval (PPI) values, coefficient of variation (CV) of PPI values, breathing amplitude, and spectral features derived by applying a Fast Fourier Transform (FFT) algorithm to the respiratory motion data.

In certain examples, the method may further comprise analyzing, by a data analyzation component, including, for example, the data analyzation component 318 of FIG. 3 or the data analyzation component 570 of FIG. 5, the extracted respiratory features to determine whether the user's current breathing pattern falls within an optimal or target range. In certain examples, the analysis may be performed with reference to the reference data library 414, which may provide population-level respiratory data organized by demographic group. This may include, for example, age group and health status for comparison with the user's real-time data.

The method may further comprise generating, by the AI-assisted controller 420, a feedback signal specifying adjustments to one or more music parameters of the music source 412 or music source 322 based on the analysis of the user's respiratory features, and transmitting the feedback signal to the music source to adjust the music output in real time. In certain examples, music parameter adjustments may include, for example, increasing or decreasing tempo to increase or decrease the user's breathing rate, adjusting volume, or changing the music selection, leveraging the observed tendency for breathing to synchronize with music tempo after approximately ten to twenty-five seconds of exposure.

Additionally, the method may further comprise detecting an anomalous breathing pattern, for example, a pattern indicative of an approaching apnea event or of an irregular breathing amplitude, by the AI-assisted controller 420 and, in response, first adjusting music parameters to attempt to restore the user's breathing to an optimal condition, and, if optimal conditions are not restored, generating an alert, including, for example, transmitting a notification to a designated contact or healthcare provider via a network-connected device.

In certain examples, the method may further comprise storing respiratory motion data and extracted respiratory features from each user session in a database to build a longitudinal respiratory health profile for the user. In certain examples, the stored data may be used as input for the machine learning component 416 to refine the machine learning models and to provide improved predictions of the user's respiratory health over time.

It should be noted that the methods described herein describe possible implementations. The operations and the steps may be rearranged, or otherwise modified, and other implementations are possible. Further, aspects from two or more of the methods may be combined.

The various illustrative blocks and components described in connection with the disclosure herein may be implemented or performed using a general-purpose processor, a DSP, an ASIC, a Central Processing Unit (CPU), a graphics processing unit (GPU), a neural processing unit (NPU), a Field Programmable Gate Array (FPGA) or other PLD, a quantum processor, a discrete gate or transistor logic, discrete hardware components, or any combination thereof designed to perform the functions described herein. A general-purpose processor may be a microprocessor, but, in the alternative, the processor may be any processor, controller, microcontroller, or state machine. A processor may also be implemented as a combination of computing devices (for example, a combination of a DSP and a microprocessor, multiple microprocessors, one or more microprocessors in conjunction with a DSP core, a combination of classical and quantum processors, or any other such configuration). Any functions or operations described herein as being capable of being performed by a processor may be performed by multiple processors that, individually or collectively, are capable of performing the described functions or operations.

The functions described herein may be implemented using hardware, software executed by one or more processors, firmware, or any combination thereof. If implemented using software executed by multiple processors, the functions may be stored as or transmitted using one or more instructions or code of a computer-readable medium. Other examples and implementations are within the scope of the disclosure and appended claims. For example, due to the nature of software, functions described herein may be implemented using software executed by one or more processors, hardware, controllers, firmware, hardwiring, circuitry, or combinations of any of these. Features implementing functions may also be physically located at various positions, including being distributed such that portions of functions are implemented at different physical locations.

Computer-readable media include both non-transitory computer storage media and communication media, including any medium that facilitates transfer of a computer program from one location to another. A non-transitory storage medium may be any available medium that may be accessed by a general-purpose or special-purpose computer, whether classical or quantum. By way of example, and not limitation, non-transitory computer-readable media may include random access memory (RAM), read-only memory (ROM), electrically erasable programmable ROM (EEPROM), flash memory, compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer or a general-purpose or special-purpose processor. Also, any connection may be properly termed a computer-readable medium. For example, the software may be transmitted from a website, server, or other remote source using a wired technology such as a coaxial cable, fiber optic cable, twisted pair, digital subscriber line (DSL), universal serial bus (USB), high-definition multimedia interface (HDMI), video graphics array (VGA), digital visual interface (DVI), thunderbolt cable, power cable, ribbon cable, integrated services digital network (ISDN), or wireless technologies such as wireless fidelity (Wi-Fi), Bluetooth, cellular network, near-field communication (NFC), Zigbee, long range (LoRa), infrared (IR), radio frequency identification (RFID), light fidelity (Li-Fi), satellite, ultra-wideband (UWB), millimeter wave (mmWave), and microwave. The wired and/or wireless technologies are included in the definition of computer-readable medium. Disk and disc, as used herein, include a compact disk (CD), laser disc, optical disc, digital versatile disc (DVD), floppy disk, and Blu-ray disc. Disks or discs may reproduce data magnetically or optically using lasers. Combinations of the above are also included within the scope of computer-readable media. Any functions or operations described herein as being capable of being performed by a memory may be performed by multiple memories that, individually or collectively, are capable of performing the described functions or operations.

As used herein, the term “computing” may refer to any operations that may be performed by a computer (or a computing device), including (but not limited to): computation, data storage, data retrieval, communication, execution of an algorithm, and the like. Further, as used herein, a “computing device” may refer to any device in which a computing operation may be carried out. A computing device may be, for example (but not limited to): a compute component, a storage component, a network device, a telecommunications component, and the like.

As used herein, the term “computing resource” may refer to any program, application, document, asset, executable program file, desktop environment, computing environment, network environment, or other resource made available to, for example, a user of a computing device. A computing resource may be delivered to a computing device via, for example (but not limited to): conventional installation, a method of streaming, a virtual machine executing on a remote computing device, execution from a removable storage device connected to the computing device (e.g., a universal serial bus (USB) device), and the like.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof. As used herein, outputting at least one signal may refer to any type of signal that may be output, including wireless communication signals, electrical signals, or any other type of signal that may be transferred by voltages, currents, electromagnetic waves, magnetic fields or particles, optical fields or particles, or any combination thereof. As used herein, “communication” may refer to data transferring or passing, or may refer to two or more components coordinating a job or task. As used herein, the term “data” is intended to be broad in scope. In this manner, the term “data” embraces, for example, (but not limited to): a data stream (or stream data), data chunks, data blocks, atomic data, objects of any type, files of any type (e.g., media files, spreadsheet files, database files, etc.), directories, sub-directories, volumes, and the like.

Machine learning includes one or more systems that may be adapted to generate, train, and execute one or more trained learning models, nodes, neural networks, gradient boosting algorithms, mutual information classifiers, random forest classifications, and other machine learning and artificial intelligence-based algorithms or models to process inputs, parameters, and other data elements. In some examples, the one or more trained learning models can include deep learning, machine learning, neural networks, computer vision, and similar advanced artificial intelligence-based technologies. In some examples, the machine learning or artificial intelligence may use additional inputs and/or feedback loops in an iterative training process for enhanced data processing and improved outcomes.

Although the disclosure may describe components and functions that may be implemented in a particular example with reference to a particular standard or protocol, the disclosure is not limited to the standard or protocol. Other standards or protocols supporting similar functionality are considered equivalents thereof.

As used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Furthermore, “and/or” as used in a list of items indicates an inclusive list such that, for example, a list of at least one of A, B, and/or C means A or B or C or AB or AC or BC or ABC (i.e., A and B and C). Also, as used herein, the phrase “based on” shall not be construed as a reference to a closed set of conditions. For example, an example step that may be described as “based on condition A” may be based on both a condition A and a condition B. For example, as used herein, the phrase “based on” shall be construed in the same manner as the phrase “based at least in part on.”

As used herein, including in the claims, the article “a” before a noun may be open-ended and understood to refer to “at least one” of those nouns or “one or more” of those nouns. Thus, the terms “a,” “at least one,” “one or more,” and “at least one of one or more” may be interchangeable. For example, if a claim recites “a component” that performs one or more functions, each of the individual functions may be performed by a single component or by any combination of multiple components. Thus, the term “a component” having characteristics or performing functions may refer to “at least one of one or more components” having a particular characteristic or performing a particular function. Subsequent reference to a component introduced with the article “a” using the terms “the” or “said” may refer to any or all of the one or more components. For example, a component introduced with the article “a” may be understood to mean “one or more components,” and referring to “the component” subsequently in the claims may be understood to be equivalent to referring to “at least one of the one or more components.” Similarly, subsequent reference to a component introduced as “one or more components” using the terms “the” may refer to any or all of the one or more components. For example, referring to “the one or more components” subsequently in the claims may be understood to be equivalent to referring to “at least one of the one or more components.”

The terms “determine,” “determining,” “identify,” or “identifying” encompass a variety of actions and, therefore, “determining” or “identifying” can include calculating, computing, processing, deriving, investigating, looking up (such as via looking up in a table, a database, or another data structure), receiving, ascertaining, and the like. Also, “determining” or “identifying” can include receiving (for example, receiving information), accessing (for example, accessing data stored in memory), retrieving, and the like. Also, “determining” or “identifying” can include resolving, obtaining, selecting, choosing, establishing, and other such similar actions.

In the appended figures, similar components or features may have the same reference label. Further, various components of the same type may be distinguished by following the reference label by a dash and a second label that distinguishes among the similar components. If just the first reference label may be used in the specification, the description may be applicable to any one of the similar components having the same first reference label irrespective of the second reference label or other subsequent reference label.

The description set forth herein, in connection with the appended figures, describes example configurations and does not represent all the examples that may be implemented or that are within the scope of the claims. The term “example” or “instance” used herein means “serving as an example, instance, or illustration” and not “preferred” or “advantageous over other examples.” The detailed description includes specific details for the purpose of providing an understanding of the described techniques. These techniques, however, may be practiced without these specific details. In some figures, known structures and devices are shown in block diagram form in order to avoid obscuring the concepts of the described examples. The features of the various examples described herein may be combined in any suitable manner. It is contemplated that one or more features from one example may be incorporated into another example unless explicitly stated otherwise. The combinations of features from different examples are within the scope of the disclosure.

Although terms such as “document,” “file,” “segment,” “block,” or “object” may be used by way of example, the present disclosure is not limited to any particular form of representing and storing data or other information. Rather, the present disclosure may be equally applicable to any object capable of representing information.

It will be appreciated by those skilled in the art that while the disclosure has been described above in connection with particular examples, the disclosure is not necessarily so limited, and that numerous other examples, uses, means for, modifications and departures from the examples, uses, and means for are intended to be encompassed by the claims attached hereto. The entire disclosure of each patent and publication cited herein is incorporated by reference, as if each such patent or publication were individually incorporated by reference herein. Various features and advantages of the disclosure are set forth in the following claims.

1. A system for music-modulated respiratory therapy, comprising:
a non-contact sensor positioned about a user, the non-contact sensor configured to detect respiratory motion based on changes in magnetic flux;
a music source configured to output audio to the user, the music source including one or more adjustable parameters; and
a controller operatively coupled to the non-contact sensor and the music source, the controller configured to:
receive respiratory motion data from the non-contact sensor;
determine a user respiratory pattern based on the respiratory motion data; and
generate a feedback signal to adjust the one or more adjustable parameters of the music source based on the user respiratory pattern.

2. The system of claim 1, wherein the controller is configured to extract one or more respiratory features from the respiratory motion data comprising at least one of a breathing rate, a breathing uniformity value, a breathing amplitude, one or more spectral features or a combination thereof.

3. The system of claim 2, wherein the controller is further configured to compare one or more extracted respiratory features to determine if a user respiratory pattern falls within a target range.

4. The system of claim 1, wherein the non-contact sensor comprises a Hall sensor configured to detect changes in magnetic flux.

5. The system of claim 1, wherein the controller incorporates one or more machine learning models configured to:
classify the user respiratory pattern; and
determine appropriate adjustments to the one or more adjustable parameters of the music source based on the classified respiratory pattern.

6. The system of claim 5, wherein the one or more machine learning models are configured to differentiate between breathing patterns associated with at least one of healthy breathing, apnea precursor events, COVID-19-associated breathing anomalies, lung disease patterns, heart disease patterns, or a combination thereof.

7. The system of claim 1, wherein the controller is further configured to:
detect an anomalous breathing pattern in the respiratory motion data;
adjust the one or more adjustable parameters of the music source;
classify a responsive breathing pattern following the adjustment;
generate and transmit an alert notification via a network-connected device if the responsive breathing pattern does not meet an improvement criterion.

8. The system of claim 1, wherein the controller is further configured to store respiratory motion data and extracted respiratory features from each user session, and to use the stored respiratory motion data to refine one or more machine learning models.

9. The system of claim 1, wherein the music source comprises a smartphone configured to serve as an adaptive music source that connected via a wireless link, and wherein the one or more adjustable parameters of the adaptive music source comprise at least one of: tempo, volume, rhythm, pitch, or music selection.

10. A computing device for music-modulated respiratory therapy, comprising:
at least one memory for storing processor-executable instructions; and
at least one processor communicatively connected with the at least one memory and individually or collectively operable to execute the instructions to cause the computing device to:
receive user information from at least one external source;
receive respiratory motion data from a non-contact sensor installed on a user;
determine a user respiratory pattern based on the respiratory motion data;
compare the respiratory motion data to the user information to determine a deviation;
adjust one or more parameters of a music source output based on the deviation.

11. The computing device of claim 10, wherein the processor-executable instructions further cause the at least one processor to extract one or more respiratory features from the respiratory motion data, the one or more respiratory features comprising at least one of, a breathing rate, a breathing uniformity value, a breathing amplitude, spectral features, or a combination thereof.

12. The computing device of claim 10, wherein the processor-executable instructions further cause the at least one processor to compare the extracted one or more respiratory features against reference values and determine whether a user current respiratory pattern falls within a target range.

13. The computing device of claim 10, wherein the processor-executable instructions further cause the at least one processor to invoke one or more machine learning models trained on respiratory data from a plurality of subjects to classify the user respiratory state, and generate predictions of appropriate adjustments to the one or more adjustable parameters of the music source.

14. The computing device of claim 13, wherein the one or more machine learning models comprise at least one of a classification algorithm, a regression algorithm, an anomaly detection algorithm, a clustering algorithm, or a combination thereof.

15. The computing device of claim 10, wherein the processor-executable instructions further cause the at least one processor to detect an anomalous breathing pattern in the respiratory motion data, generate a feedback signal to adjust the one or more adjustable parameters of the music source, classify a responsive breathing pattern following the adjustment, and generate and transmit an alert notification via a network-connected device if the responsive breathing pattern does not meet an improvement criterion.

16. The computing device of claim 10, wherein the processor-executable instructions further cause the at least one processor to store respiratory motion data and extracted respiratory features from each user session in a database to build a health profile for the user, and refine one or more machine learning models over time based on the health profile.

17. The computing device of claim 10, wherein the processor-executable instructions further cause the at least one processor to map the respiratory motion data to a time-series representation of a user breathing pattern, and extract the one or more respiratory features.

18. The computing device of claim 10, wherein the adjustable parameters of the music source comprise at least one of: tempo, volume, rhythm, pitch, or music selection, and wherein the processor-executable instructions further cause the at least one processor to decrease the tempo of music output to induce a deceleration of a user breathing rate, or to increase the tempo of music output to increase the user breathing rate, based on the user respiratory pattern.

19. The computing device of claim 10, wherein the computing device comprises a smartphone, and wherein the processor-executable instructions further cause the at least one processor to receive respiratory motion data from the non-contact sensor via a wireless link and to process the respiratory motion data in real time to generate a feedback signal adjusting the one or more adjustable parameters of the music source.

20. A method for a music-modulated respiratory therapy system, comprising:
receiving user information from at least one external source;
installing a non-contact sensor to a user;
receiving respiratory motion data from a non-contact sensor;
determining a user respiratory pattern based on the respiratory motion data;
comparing the respiratory motion data to the user information to determine a deviation; and
adjusting one or more parameters of a music source output based on the deviation.
